(19) United States
(12) Patent Application Publication
(10) Pub. No.: US 2013/0237840 A1
(43) Pub. Date: Sep. 12, 2013

(54) Dressing for an Electromagnetic Spectrum Sensor

(71) Applicants: Gary P Warren, (US); Marilyn J. Bowers, (US); Matthew S. Alley, (US); Scott J. Anchell, (US); IVWATCH, LLC, Williamsburg, VA (US)

(72) Inventors: Gary P. Warren, Williamsburg, VA (US); Marilyn J. Bowers, Toano, VA (US); Matthew S. Alley, Sandston, VA (US); Scott J. Anchell, Fairfax Station, VA (US)

(73) Assignee: IVWATCH, LLC, Williamsburg, VA (US)

(21) Appl. No.: 13/794,776

(22) Filed: Mar. 11, 2013

Related U.S. Application Data
(60) Provisional application No. 61/609,865, filed on Mar. 12, 2012.

(51) Int. Cl. A61B 5/00

(52) U.S. Cl. CPC A61B 5/441; A61B 5/0082
USPC 600/473

(57) ABSTRACT

An epidermal dressing includes a barrier film and a fitting that cooperates with a sensor emitting and detecting near infrared signals for monitoring an intravascular infusion. The barrier film overlies an epidermal insertion site for a cannula administering the intravascular infusion. The fitting includes a first arrangement that retains the sensor and a second arrangement that releases the sensor from the first arrangement.

### CROSS-REFERENCES TO RELATED APPLICATIONS

This application claims the priority of U.S. Provisional Application No. 61/609,865, filed 12 Mar. 2012, which is hereby incorporated by reference in its entirety.

### STATEMENT REGARDING FEDERALLY SPONSORED RESEARCH OR DEVELOPMENT

Not Applicable

### BACKGROUND OF THE INVENTION

FIG. 10 shows a typical arrangement for intravascular infusion. The term intravascular preferably refers to being situated in, occurring in, or being administered by entry into a blood vessel, thus “intravascular infusion” preferably refers to introducing a fluid into a blood vessel. Intravascular infusion accordingly encompasses both intravenous infusion (administering a fluid into a vein) and intra-arterial infusion (administering a fluid into an artery).

A cannula 20 is typically used for administering fluid via a subcutaneous blood vessel. Typically, cannula 20 is inserted through epidermis E at an insertion site S and punctures, for example, the cephalic vein, basilica vein, median cubital vein, or any suitable vein for an intravenous infusion. Similarly, any suitable artery may be used for an intra-arterial infusion.

Cannula 20 typically is in fluid communication with a fluid source 22. Typically, cannula 20 includes a hub 20a or another extracorporeal connector and fluid source 22 includes one or more sterile containers that hold the fluid(s) to be administered. Examples of typical sterile containers include plastic bags, glass bottles or plastic bottles.

An administration set 30 typically provides a sterile conduit for fluid to flow from fluid source 22 to cannula 20. Typically, administration set 30 includes tubing 32, a drip chamber 34, a flow control device 36, and a cannula connector 38. Tubing 32 is typically made of polypropylene, nylon, or another flexible, strong and inert material. Drip chamber 34 typically permits the fluid to flow one drop at a time for reducing air bubbles in the flow. Tubing 32 and drip chamber 34 are typically transparent or translucent to provide a visual indication of the flow. Typically, flow control device 36 is positioned upstream from drip chamber 34 for controlling fluid flow in tubing 34. Roller clamps and Dial-A-Flo®, manufactured by Hospira, Inc. (Lake Forest, Ill., USA), are examples of typical flow control devices. Typically, cannula connector 38 and hub 20a provide a leak-proof coupling through which the fluid may flow. Luer-Lok™, manufactured by Becton, Dickinson and Company (Franklin Lakes, N.J., USA), is an example of a typical leak-proof coupling.

Administration set 30 may also include at least one of a clamp 40, an injection port 42, a filter 44, or other devices. Typically, clamp 40 pinches tubing 34 to cut-off fluid flow. Injection port 42 typically provides an access port for administering medicine or another fluid via cannula 20. Filter 44 typically purifies and/or treats the fluid flowing through administration set 30. For example, filter 44 may strain contaminants from the fluid.

An infusion pump 50 may be coupled with administration set 30 for controlling the quantity or the rate of fluid flow to cannula 20. The Alaris® System manufactured by CareFusion Corporation (San Diego, Calif., USA) and Flo-Gard® Volumetric Infusion Pumps manufactured by Baxter International Inc. (Deerfield, Ill., USA) are examples of typical infusion pumps.

Unintended infusing typically occurs when fluid from cannula 20 escapes from its intended vein/artery. Typically, unintended infusing causes an abnormal amount of a substance to diffuse or accumulate in perivascular tissue or cells and may occur, for example, when (i) cannula 20 causes a brittle vein/artery to rupture; (ii) cannula 20 improperly punctures the vein/artery; (iii) cannula 20 is improperly sized; or (iv) infusion pump 50 administers fluid at an excessive flow rate. Unintended infusing of a non-vesicant fluid is typically referred to as “infiltration,” whereas unintended infusing of a vesicant fluid is typically referred to as “extravasation.”

The symptoms of infiltration or extravasation typically include blanching or discoloration of the epidermis E, edema, pain, or numbness. The consequences of infiltration or extravasation typically include skin reactions such as blisters, nerve compression, acute limb compartment syndrome, or necrosis. Typical care for infiltration or extravasation includes applying warm compresses, administering hyaluronidase or phentolamine, fasciotomy, or amputation.

### BRIEF SUMMARY OF THE INVENTION

Embodiments according to the present invention include a dressing for coupling an electromagnetic spectrum sensor and an epidermis. The electromagnetic spectrum sensor is configured to monitor an intravascular infusion. The dressing includes a fitting and a frame coupled to the fitting. The fitting includes a pocket. The fitting has a first arrangement configured to retain the electromagnetic spectrum sensor in the pocket and a second arrangement configured to release the electromagnetic spectrum sensor from the first arrangement. The frame is configured to overlay an area of the epidermis that is larger than that overlaid by the fitting.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features, principles, and methods of the invention.

FIG. 1 is a schematic view illustrating an embodiment of a dressing according to the present disclosure.

FIG. 2 is a partially exploded schematic cross-section view of the dressing shown in FIG. 1.

FIGS. 3A-3D illustrate a fitting of the dressing shown in FIG. 1. FIG. 3A is a plan view, FIG. 3B is a cross-section view taken along line IIIB-IIIB in FIG. 3A, FIG. 3C is an enlarged view illustrating detail IIIC in FIG. 3B, and FIG. 3D is an enlarged view illustrating detail IIID in FIG. 3B.

FIG. 4 is a schematic view illustrating an embodiment of a dressing according to the present disclosure.

FIGS. 5A-5D are schematic views illustrating details of the dressing shown in FIG. 4. FIG. 5A is a cross-section view taken along line VA-VA in FIG. 4 with an electromagnetic spectrum sensor shown in dash-dot line, FIG. 5B is a detail view showing features of the electromagnetic spectrum sensor in FIG. 5A, FIG. 5C is a cross-section view taken along line VC-VC in FIG. 4, and FIG. 5D is a cross-section view taken along line VD-VD in FIG. 4.

FIGS. 6A and 6B are schematic views illustrating alternate dressings of an embodiment according to the present disclosure.

FIGS. 7A-7D illustrate an embodiment of a dressing according to the present disclosure. FIG. 7A is a schematic plan view showing an assembly including a contamination barrier and a frame, FIG. 7B is a schematic plan view showing the contamination barrier prior to assembly, FIG. 7C is a schematic plan view showing the frame and a lead management system prior to assembly, and FIG. 7D is a schematic plan view showing an implementation of an assembly including the contamination barrier, the frame, and the lead management system.

FIGS. 8A-8D illustrate alternate dressings of an embodiment according to the present disclosure. FIG. 8A is a schematic plan view illustrating a dressing including a fitting integrally molded with a frame, FIG. 8B is a cross-section view taken along line VIIIB-VIIIB in FIG. 8A, FIG. 8C is a schematic plan view illustrating a dressing including a fitting over-molded with a frame, and FIG. 8D is a cross-section view taken along line VIIID-VIIID in FIG. 8C.

FIG. 9 is a schematic view illustrating an embodiment of a dressing according to the present disclosure.

FIG. 10 is a schematic view illustrating a typical set-up for infusion administration.

In the figures, the thickness and configuration of components may be exaggerated for clarity. The same reference numerals in different figures represent the same component. The broken lines in the figures are for illustrative purposes only and form no part of the claimed invention.

### DETAILED DESCRIPTION OF THE INVENTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various features are described which may be included in some embodiments but not other embodiments.

The terms used in this specification generally have their ordinary meanings in the art, within the context of the disclosure, and in the specific context where each term is used. Certain terms in this specification may be used to provide additional guidance regarding the description of the disclosure. It will be appreciated that a feature may be described more than one-way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term.

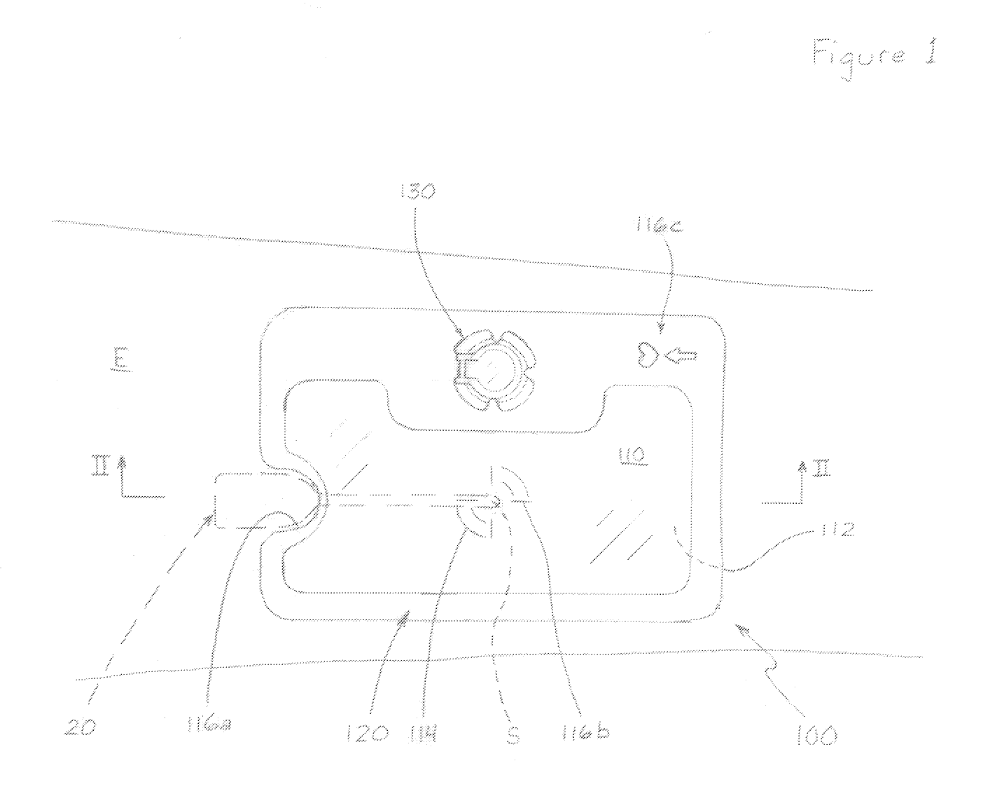

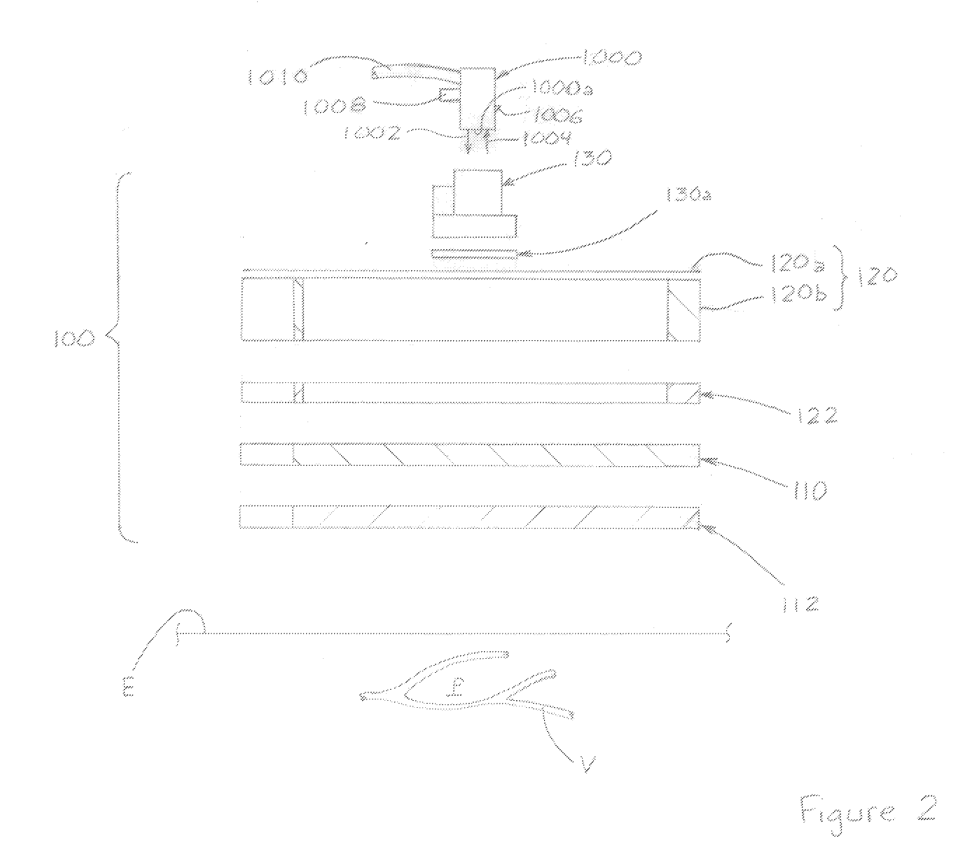

FIGS. 1 and 2 show an embodiment of a dressing 100 that includes (i) a contamination barrier overlying the insertion site S; and (ii) a fitting for coupling an electromagnetic spectrum sensor 1000 that senses if fluid is infusing perivascular tissue around cannula 20. Dressing 100 preferably provides a contamination barrier that is substantially impervious to solids, liquids, microorganisms and/or viruses. Preferably, dressing 100 may be semi-permeable to allow air or vapor to pass, thus permitting the epidermis E to breathe.

Electromagnetic spectrum sensor 1000 preferably aids in diagnosing infiltration or extravasation. Preferably, electromagnetic radiation 1002 is emitted via a sensor surface 1000a of electromagnetic spectrum sensor 1000 and electromagnetic radiation 1004 is received via sensor surface 1000a. Emitted electromagnetic radiation 1002 passes through the epidermis E into the perivascular tissue P. Referring to FIG. 2, the perivascular tissue P in the vicinity of a blood vessel V preferably includes the cells or interstitial compartments that may become unintentionally infused, e.g., infiltrated or extravasated by fluid from cannula 20. Received electromagnetic radiation 1004 is at least a portion of emitted electromagnetic radiation 1002 that is reflected, scattered, diffused, or otherwise redirected from the perivascular tissue P through the epidermis E to sensor surface 1000a.

Emitted and received electromagnetic radiations 1002 and 1004 are preferably in the near-infrared portion of the electromagnetic spectrum. The term “near infrared” refers to electromagnetic radiation having wavelengths between approximately 1,400 nanometers and approximately 700 nanometers—proximate the nominal edge of red light in the visible light portion of the electromagnetic spectrum. These wavelengths correspond to a frequency range of approximately 215 terahertz to approximately 430 terahertz.

Electromagnetic spectrum sensor 1000 may be coupled to a processor (not shown) via a lead 1010. Preferably, the processor or another suitable device analyzes changes over time in received electromagnetic radiation 1004 for providing an indication of fluid infusing the perivascular tissue P. According to other embodiments, electromagnetic spectrum sensor 1000 and the processor may be coupled wirelessly rather than via lead 1010, or electromagnetic spectrum sensor 1000 may incorporate the processor.

Dressing 100 preferably includes a pane 110 for viewing the insertion site S. Preferably, pane 110 is transparent or translucent to light in the visible portion of the electromagnetic spectrum, for example, light having wavelengths between approximately 380 nanometers and approximately 760 nanometers. These wavelengths correspond to a frequency range of approximately 400 terahertz to approximately 790 terahertz. Pane 110 preferably includes polyurethane film or another suitable material and/or construction for providing a contamination barrier that may be transparent or translucent.

An adhesive 112 preferably bonds pane 110 to the epidermis E around the insertion site S. Preferably, adhesive 112 includes an acrylic adhesive that is suitable for contact with the epidermis E or another medical grade adhesive that is biocompatible according to Standard 10993 promulgated by the International Organization for Standardization (ISO 10993) and/or Class VI promulgated by The United States Pharmacopeial Convention (USP Class VI). Adhesive 112 may be applied to pane 110 on the entire surface that confronts the epidermis E, or adhesive 112 may be omitted from one or more portions of the surface. Also, the strength of the bond between pane 110 and the epidermis E may vary according to different embodiments of dressing 100. For example, stronger or more adhesive 112 may be used for coupling dressing 100 to relatively robust skin, e.g., adult skin, and weaker or less adhesive 112 may be used for coupling dressing 100 to relatively delicate skin, e.g., pediatric skin.

Pane 110 may also include a diagnostic tool 114 to assist in visually analyzing symptoms of infiltration or extravasation. For example, diagnostic tool 114 may include a set of concentric arcs, a geometric shape, a set of parallel lines, a color gradient, or another suitable reticle for evaluating conditions at the epidermis E that may be symptomatic of infiltration or extravasation. According to one embodiment, the appearance of a set of concentric arcs or a geometric shape may become distorted when the epidermis E, and thus pane 110, is distended due to edema. According to another embodiment, changes in the coloration of the epidermis E may be evaluated by periodic comparison with a color gradient included on pane 110.

Dressing 100 is preferably located or oriented with respect to at least one of cannula 20, the insertion site S, or an anatomical feature. According to one embodiment, dressing 100 may include a notch 116a or another suitable guide that is sized or shaped for cooperating with at least a portion of cannula 20. According to another embodiment, pane 110 may include crosshairs 116b or another suitable guide for locating dressing 100 relative to the insertion site S. According to another embodiment, indicia, symbols, and/or other markings may provide a guide for relatively orienting dressing 100 with resect to an anatomical feature. For example, guide 116c includes an arrow and a symbol that suggests orienting dressing 100 upstream from the heart.

Dressing 100 preferably includes a frame 120 coupled to pane 110. Frame 120 preferably has greater resistance to deformation than does pane 110. Accordingly, frame 120 may maintain the general shape of pane 110 while dressing 100 is laid over the insertion site S. According to one embodiment, frame 120 entirely cinctures pane 110. According to other embodiments, frame 120 may (i) partially cincture pane 110; (ii) extend from a peripheral portion of pane 110 toward an interior portion of pane 110; (iii) extend from the interior portion toward the peripheral portion; (iv) be spaced from the peripheral portion; or (v) include a combination of (i)-(iv). Frame 120 preferably includes polyvinyl chloride, polyethylene, polypropylene, or another suitable material that is relatively rigid with respect to pane 110. According to one embodiment, frame 120 may include polyethylene tape 120a being relatively associated with or disposed on a pad of polyvinyl chloride foam 120b.

Frame 120 is preferably transparent or translucent to visible light for viewing the epidermis E in the vicinity of the insertion site S. Preferably, frame 120 absorbs or blocks the transmission of radiation having the same wavelength as emitted electromagnetic radiation 1002, e.g., near infrared radiation. Thus, according to one embodiment, the epidermis E that underlies frame 120 may be optically visible and shielded from ambient near-infrared radiation.

Frame 120 is preferably coupled to pane 110 by an adhesive 122 or another suitable coupling. According to one embodiment, adhesive 122 preferably provides a coupling between pane 110 and frame 120 that is relatively stronger than the bond between pane 110 and the epidermis E. Accordingly, pane 110 remains attached to frame 120 when separating dressing 100 from the epidermis E. Adhesive 122 according to another embodiment of dressing 100 preferably provides a coupling between pane 110 and frame 120 that is relatively weaker than the bond between pane 110 and the epidermis E. Accordingly, frame 120 may be released from pane 110 after dressing 100 is laid over the insertion site S.

Dressing 100 preferably includes a fitting 130 for coupling an anatomic sensor with the epidermis E. As the terminology is used herein, “anatomic” preferably refers to the structure of a body and an “anatomic sensor” preferably is concerned with sensing a change over time of the structure of the body. By comparison, a physiological sensor is concerned with sensing the functions and activities of a body, e.g., pulse, at a point in time.

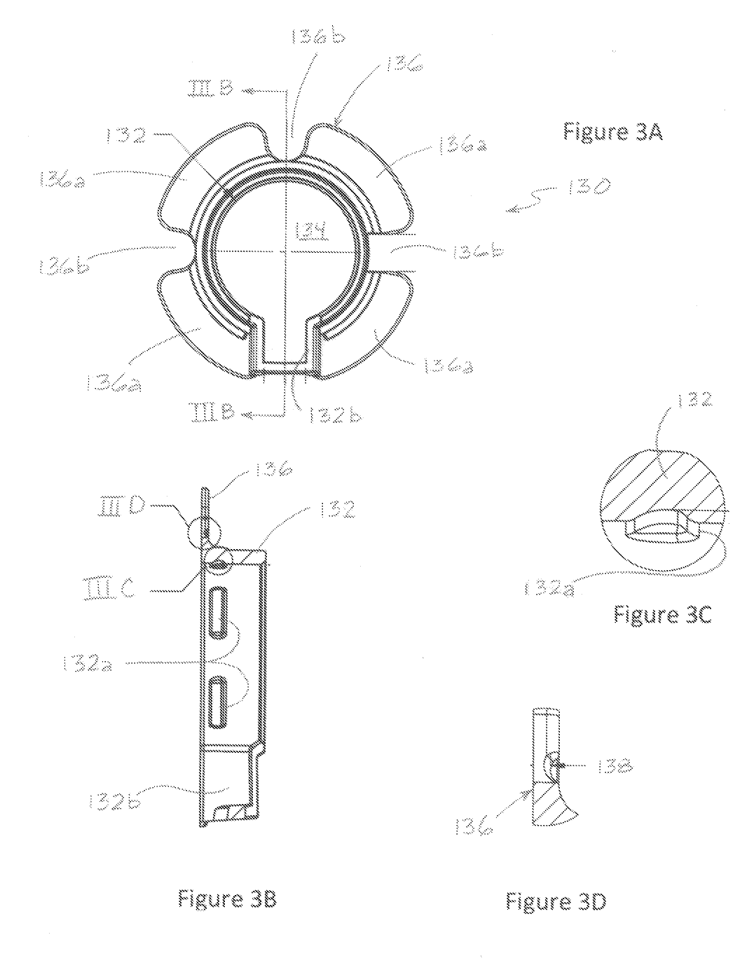

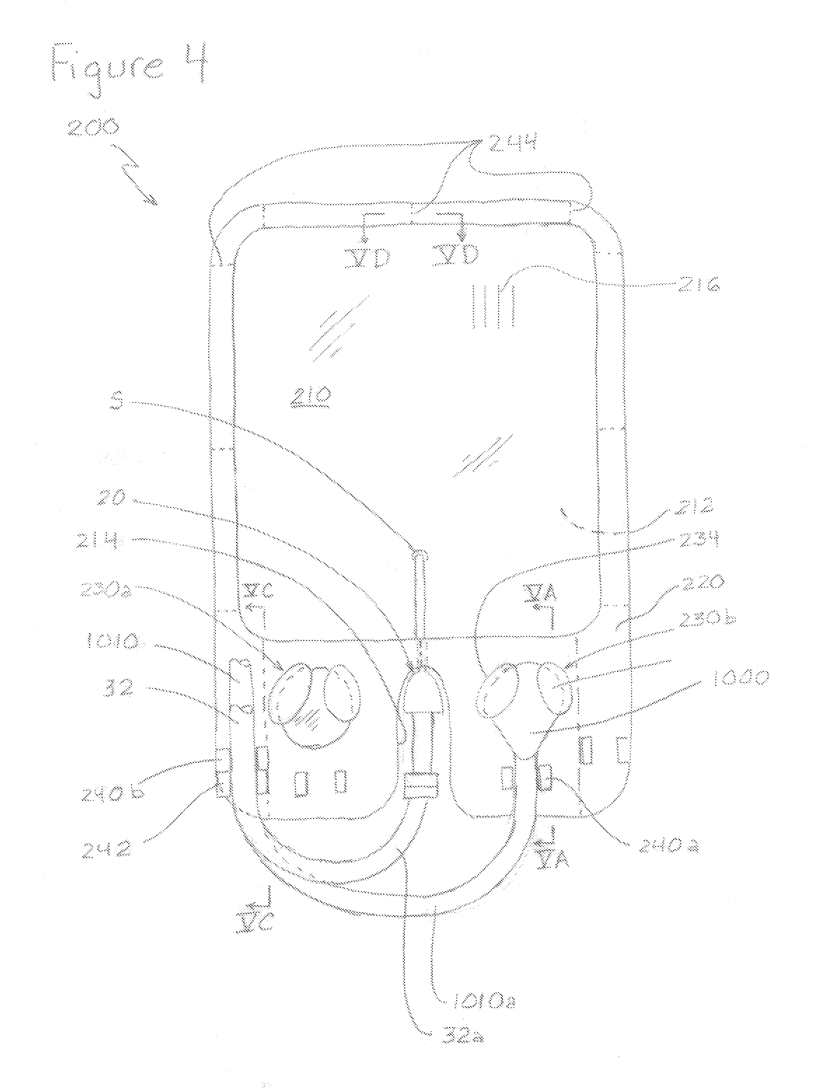

There are preferably two arrangements of fitting 130 with respect to electromagnetic spectrum sensor 1000. The term “arrangement” as it is used herein preferably refers to a relative configuration, formation, layout or disposition of fitting 130 and electromagnetic spectrum sensor 1000. A first arrangement of fitting 130 preferably retains electromagnetic spectrum sensor 1000 relative to dressing 100 for monitoring infiltration or extravasation during an infusion with cannula 20. Accordingly, the first arrangement of fitting 130 with respect to electromagnetic spectrum sensor 1000 preferably senses over time if fluid from cannula 20 is infusing the perivascular tissue P. A second arrangement of fitting 130 preferably releases electromagnetic spectrum sensor 1000 from the first arrangement. The first arrangement preferably includes one or more first surfaces 1006 on electromagnetic spectrum sensor 1000 being snapped under a second surface 132a (see FIGS. 3B and 3C) on fitting 130. Accordingly, the second arrangement preferably includes snapping the first surface 1006 over the second surface 132a to release electromagnetic spectrum sensor 1000 from the first arrangement. Other embodiments may use a latch, a cap, a resilient element, or another suitable device that, in the first arrangement, retains electromagnetic spectrum sensor 1000 in fitting 130 and preferably biases sensor surface 1000a toward the epidermis E and, in the second arrangement, releases electromagnetic spectrum sensor 1000 from fitting 130, e.g., allowing electromagnetic spectrum sensor 1000 to separate from fitting 130. Accordingly, the first and second arrangements permit electromagnetic spectrum sensor 1000 to be reused with a plurality of dressings 100 that are individually applied to patients' epidermises.

Fitting 130 may be indirectly or directly coupled to pane 110. According to one embodiment of dressing 100, frame 120 preferably couples fitting 130 to pane 110. According to another embodiment of dressing 100, fitting 130 and pane 110 are preferably directly coupled. Fitting 130 is preferably fixed to dressing 100 using an adhesive 130a or another suitable coupling that is relatively stronger than the bond between pane 110 and the epidermis E. Moreover, adhesive 130a preferably couples fitting 130 to frame 120 and provides a coupling that is at least as strong as the coupling between frame 120 and pane 110.

Details according to one embodiment of fitting 130 are shown in FIGS. 3A-3D. Preferably, fitting 130 includes a wall 132 that defines a pocket 134 for receiving electromagnetic spectrum sensor 1000. In the first arrangement of fitting 130, wall 132 may (i) entirely surround electromagnetic spectrum sensor 1000; (ii) include a plurality of individual segments or posts intermittently disposed around electromagnetic spectrum sensor 1000; or (iii) have any suitable configuration for locating electromagnetic spectrum sensor 1000 with respect to dressing 100. Wall 132 preferably includes one or more second surfaces 132a—three are shown in FIG. 3B—that cooperate with first surface(s) 1006 for retaining electromagnetic spectrum sensor 1000 in pocket 134 in the first arrangement of fitting 130. Preferably, fitting 130 maintains electromagnetic spectrum sensor 1000 in a desired orientation with respect to dressing 100. According to one embodiment, fitting 130 includes a recess 132b that, in the first arrangement, cooperatively receives a projection 1008 (see FIG. 2) on electromagnetic spectrum sensor 1000. According to other embodiments, fitting 130 and electromagnetic spectrum sensor 1000 may include any suitable mating features for eliminating or at least minimizing rotation of electromagnetic spectrum sensor 1000 in pocket 134.

Fitting 130 and dressing 100 are preferably coupled via an interface that permits dressing 100 to approximately conform to epidermis E. Preferably, a rim or flange 136 projects from wall 134 and provides a surface for adhesive 130a at the interface between fitting 130 and dressing 100. According to one embodiment, flange 136 may include a plurality of segments 136a—four are shown in FIG. 3A—separated by individual gaps 136b—three are shown in FIG. 3A. One or more lines of weakness 138 may be disposed on flange 136 to increase flexibility of the interface between fitting 130 and dressing 100. Accordingly, fitting 130 may approximately conform to the contours of epidermis E to thereby facilitate, in the first arrangement, maintaining and orienting electromagnetic spectrum sensor 1000 relative to insertion site S.

Dressing 100 preferably combines in a single unit an occlusive barrier and a retainer for an anatomical sensor. According to one embodiment, the anatomical sensor may include electromagnetic spectrum sensor 1000 or another sensor for sensing over time a change of body structure, e.g., infiltration and extravasation. Preferably, the occlusive barrier includes pane 110 for protecting the insertion site S and the retainer includes fitting 130 for positioning electromagnetic spectrum sensor 1000 to sense if fluid is infusing the perivascular tissue P. Fitting 130 preferably permits electromagnetic spectrum sensor 1000 to be decoupled and recoupled with dressing 100, or decoupled from a first dressing and coupled to a second dressing. Dressing 100 preferably also includes frame 120 for distributing forces over a larger area of the epidermis E. For example, forces due to pulling or snagging lead 1010 may be distributed by pane 110, frame 120 and fitting 130 over an area of the epidermis E that is larger than that overlaid by sensor surface 1000a. Dressing 100 therefore preferably enhances an approximately consistent positional relationship between electromagnetic spectrum sensor 1000 and the perivascular tissue P when sensing infiltration or extravasation. Dressing 100 is advantageous at least because applying an occlusive dressing for an intravascular infusion concurrently establishes an approximately consistent location for an infiltration/extravasation sensor.

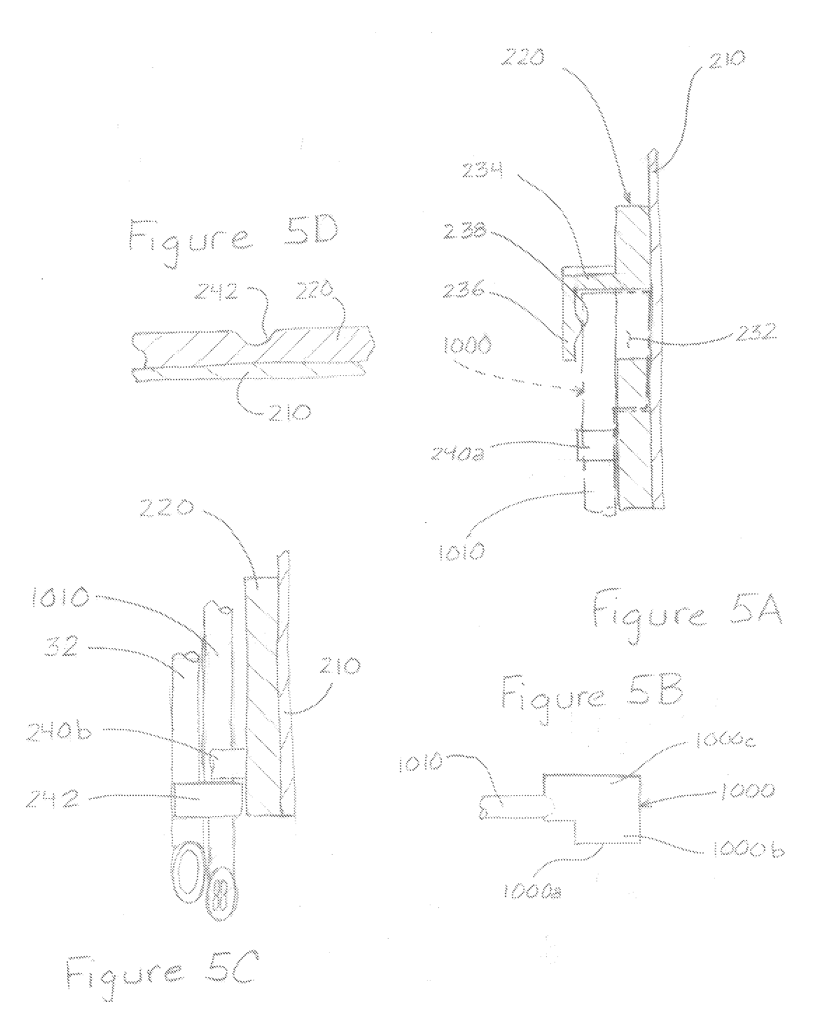

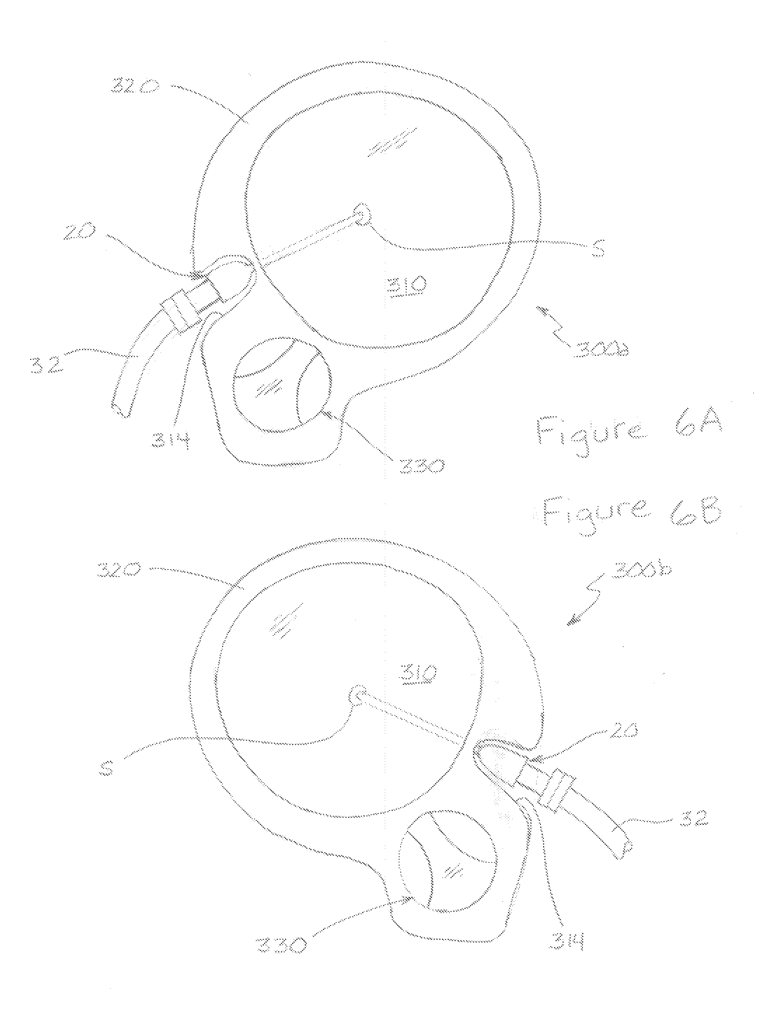

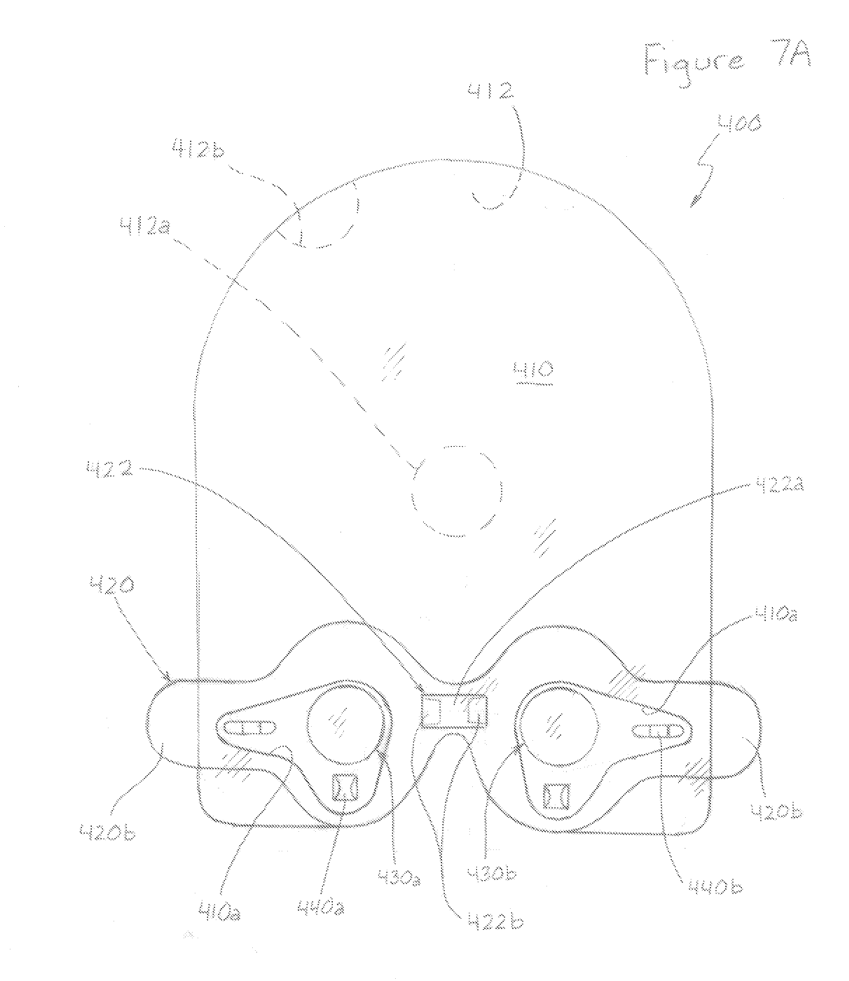

FIGS. 4 and 5A-5D show an embodiment of a dressing 200 that includes (i) a contamination barrier overlying the insertion site S; and (ii) a plurality of location options for coupling electromagnetic spectrum sensor 1000 to sense if fluid is infusing the perivascular tissue P around cannula 20. The contamination barrier preferably is substantially impervious to solids, liquids, microorganisms and/or viruses. Preferably, dressing 200 may be semi-permeable to allow air or vapor to pass, thus permitting the epidermis E to breathe.

The contamination barrier of dressing 200 preferably includes a pane 210 for viewing the insertion site S. Preferably, pane 210 is transparent or translucent to light in the visible portion of the electromagnetic spectrum. Pane 210 preferably includes a polyurethane film or another suitable material and/or construction for providing a contamination barrier that may be transparent or translucent.

An adhesive 212 preferably bonds pane 210 to the epidermis E (not indicated in FIG. 4) around the insertion site S. Preferably, adhesive 212 includes an acrylic adhesive that is suitable for contact with the epidermis E or another medical grade adhesive that is biocompatible according ISO 10993 and/or USP Class VI. Adhesive 212 may be applied to pane 210 on the entire surface that confronts the epidermis E, or adhesive 212 may be omitted from one or more portions of the surface. Also, the strength of the bond between pane 210 and the epidermis E may vary according to different embodiments of dressing 200. For example, stronger or more adhesive 212 may be used for coupling dressing 200 to relatively robust skin, e.g., adult skin, and weaker or less adhesive 212 may be used for coupling dressing 200 to relatively delicate skin, e.g., pediatric skin.

Pane 210 may also include a diagnostic tool 214 to assist in visually analyzing symptoms of infiltration or extravasation. For example, diagnostic tool 214 may include a set of concentric arcs, a geometric shape, a set of parallel lines, a color gradient, or another suitable reticle for evaluating conditions at the epidermis E that may be symptomatic of infiltration or extravasation. According to one embodiment, the appearance of a set of concentric arcs or a geometric shape may become distorted when the epidermis E, and thus pane 210, is distended due to edema. According to another embodiment, changes in the coloration of the epidermis E may be evaluated by periodic comparison with a color gradient included on pane 210.

Pane 210 may include one or more guides for positioning or orienting dressing 200 on the epidermis E. According to one embodiment, guide 216 preferably includes a notch or some other feature of dressing 200 that may be sized or shaped to receive a portion of cannula 20.

Dressing 200 preferably includes a frame 220 coupled to pane 210. According to one embodiment of dressing 200, a coupling between pane 210 and frame 220 is preferably relatively stronger than the bond between pane 210 and the epidermis E. Accordingly, pane 210 remains attached to frame 220 when separating dressing 200 from the epidermis E.

Frame 220 preferably has greater resistance to deformation than does pane 210. Accordingly, frame 220 may maintain the shape of pane 210 while dressing 200 is laid over the insertion site S. According to one embodiment, frame 220 entirely cinctures pane 210. According to other embodiments, frame 220 may (i) partially cincture pane 210; (ii) extend from a peripheral portion of pane 210 toward an interior portion of pane 210; (iii) extend from the interior portion toward the peripheral portion; (iv) be spaced from the peripheral portion; or (v) include a combination of (i)-(iv). Frame 220 preferably includes polyvinyl chloride, polyethylene, polypropylene, or another suitable material that is relatively rigid with respect to pane 210. For example, frame 220 may include a pad of polyvinyl chloride foam. Frame 220 may be opaque, but is preferably transparent or translucent to visible light for viewing the epidermis E in the vicinity of the insertion site S. Preferably, frame 220 absorbs or blocks the transmission of radiation having the same wavelength as emitted electromagnetic radiation 1002, e.g., near infrared radiation. Thus, according to one embodiment, the epidermis E that underlies frame 220 may be optically visible and shielded from ambient near-infrared radiation.

Dressing 200 preferably includes a plurality of fittings to provide alternate location options for coupling with electromagnetic spectrum sensor 1000 to dressing 200. Preferably, first fitting 230a and second fitting 230b are disposed at locations on opposite sides of guide 216. Accordingly, the first arrangements of first and second fittings 230a and 230b preferably include location options for retaining electromagnetic spectrum sensor 1000 on either side of guide 216 for monitoring infiltration or extravasation during an infusion with cannula 20. Second arrangements of first fitting 230a and second fitting 230b preferably release electromagnetic spectrum sensor 1000 from the first arrangements for the respective fittings.

Dressing 200 preferably includes multiple fittings to permit multiple options for locating electromagnetic spectrum sensor 1000 relative to the insertion site S. Preferably, electromagnetic spectrum sensor 1000 may be disposed in one of first and second fittings 230a and 230b with the other of first and second fittings 230a and 230b may be used for controlling tubing 32 and/or lead 1010. Permutations of the arrangements of first and second fittings 230a and 230b with respect to electromagnetic spectrum sensor 1000 may be characterized as “conditions” of dressing 200. For example, a first condition of dressing 200 may be characterized by the second arrangements of first and second fittings 230a and 230b. Accordingly, electromagnetic spectrum sensor 1000 is not coupled to dressing 200 in the first condition. Electromagnetic spectrum sensor 1000 may be moved from the first condition to a second condition of dressing 200 so as to be in the first arrangement of the first fitting 230a and in the second arrangement of second fitting 230b. Accordingly, electromagnetic spectrum sensor 1000 would be retained in first fitting 230a on the left-hand side of guide 216 as viewed in FIG. 4. Electromagnetic spectrum sensor 1000 may also be moved from the first condition to a third condition of dressing 200 so as to be in the first arrangement of the second fitting 230b and in the second arrangement of first fitting 230a. Accordingly, electromagnetic spectrum sensor 1000 would be retained in second fitting 230b on the right-hand side of guide 216 as viewed in FIG. 4. Dressing 200 may also be changed between the second and third conditions—moving electromagnetic spectrum sensor 1000 to the other side of guide 216—and may also be changed from either of the second or third conditions to the first condition—decoupling electromagnetic spectrum sensor 1000. Accordingly, electromagnetic spectrum sensor 1000 may be used and reused with a plurality of individual dressings 200 and on whichever side of guide 216 is advantageous for a particular patient or a particular insertion site S. Factors for evaluating which of first and second fittings 230a and 230b may be advantageous to use for retaining electromagnetic spectrum sensor 1000 preferably include reducing the likelihood of pulling or snagging lead 1010, properly placing electromagnetic spectrum sensor 1000 relative to the insertion site 2, or patient comfort.

Referring additionally to FIG. 5A, individual fittings preferably are each capable of retaining electromagnetic spectrum sensor 1000. Preferably, individual fittings, e.g., first fitting 230a or second fitting 230b, each include a pocket 232 that is defined by a wall 234. Pocket 232 preferably receives electromagnetic spectrum sensor 1000 (shown in dash-dot line in FIG. 5A) in the first arrangement. Preferably, pane 210 extends across pocket 232 and is interposed between sensor surface 1000a and the epidermis E in the first arrangement, as shown in, e.g., FIG. 5A. According to one embodiment, wall 234 preferably includes a plurality of individual segments disposed partially around pocket 232. Preferably, at least one tab 236 projects from wall 234 and overlies a portion of electromagnetic spectrum sensor 1000 in the first arrangement. Elastic deformation of wall 234 or tab 236 preferably permits electromagnetic spectrum sensor 1000 to snap-in to pocket 232 in the first arrangement and to snap-out from pocket 232 in the second arrangement. According to one embodiment, tab 236 preferably includes a raised portion or bump 238 for biasing sensor surface 1000a toward the epidermis E by contiguously engaging electromagnetic spectrum sensor 1000 in the first arrangement. According to other embodiments, individual fittings may include a latch, a cap, a resilient element, or another suitable device which, in a first arrangement, retains electromagnetic spectrum sensor 1000 in pocket 232 and preferably biases sensor surface 1000a toward the epidermis E, and in a second arrangement, releases electromagnetic spectrum sensor 1000 to move out of pocket 232.

Referring additionally to FIG. 5B, electromagnetic spectrum sensor 1000 and individual fittings in the first arrangement preferably are coupled in a desired manner. Preferably, a portion of electromagnetic spectrum sensor 1000 has a first feature that cooperates with a second feature of pocket 232. According to one embodiment, electromagnetic spectrum sensor 1000 includes a front-side cylindrical portion 1000b having a first cross-section shape and pocket 232 has a second cross-section shape that matingly receives front-side cylindrical portion 1000b. Preferably, the first and second cross-sectional shapes are approximately congruent circles or other suitable mating shapes. Portions of electromagnetic spectrum sensor 1000 other than front-side cylindrical portion 1000b preferably do not fit in pocket 232. According to one embodiment, electromagnetic spectrum sensor 1000 preferably includes a backside cylindrical portion 1000c having a third cross-section shape, e.g., a tear drop shape, that does not matingly cooperate with the second feature of pocket 232. Accordingly, electromagnetic spectrum sensor 1000 preferably can matingly engage individual fittings in only one manner.

Referring additionally to FIG. 5C, strain relief devices preferably redirect forces from lead 1010 to dressing 200. Preferably, individual fittings, e.g., first fitting 230a or second fitting 230b, each include a set of strain relief devices that contiguously engage lead 1010 in the first arrangement. According to one embodiment, each set of strain relief devices preferably includes a first fixture 240a and a second fixture 240b. Individual fixtures 240a or 240b preferably each include a pair of posts separated by a gap that is smaller than the diameter of lead 1010. Accordingly, lead 1010 may be retained by an interference fit between a pair of posts that preferably limit lateral and/or axial movement of lead 1010 relative to frame 220.

Preferably, first and second fixtures 240a and 240b are disposed on opposite sides of guide 216. In the first arrangement, first fixture 240a preferably retains lead 1010 proximate a first one of the first and second fittings 230a and 230b, and second fixture 240b preferably retains lead 1010 and tubing 32 proximate a second one of the first and second fittings 230a and 230b. First fixture 240a of second fitting 230b is shown on the right-hand side of guide 216 as viewed in FIG. 4 and second fixture 240b of second fitting 230b is shown on the left-hand side of guide 216 as viewed in FIG. 4. According to one embodiment, first fixture 240a preferably cooperates with lead 1010 to eliminate or at least minimize rotation of electromagnetic spectrum sensor 1000 in pocket 232, and second fixture 240b preferably establishes a first bight 1010a and a second bight 32a for lead 1010 and tubing 32, respectively.

Dressing 200 includes substantially identical features at different location options to increase compatibility of a single dressing for individual patients' cases. Preferably, multiple fittings and fixtures permit selecting the best available option for positioning electromagnetic spectrum sensor 1000 relative to the insertion site S and for controlling lead 1010 and/or tubing 32. Selecting either first fitting 230a or second fitting 230b preferably reduces the likelihood of pulling or snagging lead 1010 and/or tubing 32, positions electromagnetic spectrum sensor 1000 proximate to the insertion site S, and increases patient comfort.

A clip 242 preferably couples tubing 32 and lead 1010. Preferably, clip 242 may be fixed to lead 1010 at a selected distance from electromagnetic spectrum sensor 1000. The distance is preferably selected to cooperate with second fixture 240b for consistently establishing an approximate size and radius of first bight 1010a. According to one embodiment, clip 242 abuts against second fixture 240b. Clip 240 preferably includes a first portion cincturing lead 1010 and a second portion having an opening for receiving and retaining, e.g., by interference fit, tubing 32. Thus, first fixture 240a, second fixture 240b, and clip 242 preferably redirect to dressing 200 rather than electromagnetic spectrum sensor 1000 or cannula 20 any forces due to pulling or snagging lead 1010 and/or tube 32. Accordingly, in the first arrangement, electromagnetic spectrum sensor 1000 may be retained in an approximately consistent positional relationship with respect to the perivascular tissue P around cannula 20 when sensing infiltration or extravasation.

Referring additionally to FIG. 5D, frame 220 preferably is sufficiently flexible to conform to the approximate contours of epidermis E. Preferably, frame 220 includes one or more lines of weakness 242 disposed about frame 220 at various positions including, for example, in the general vicinity of corners for pane 210 and parallel to the longitudinal axis of cannula 20. According to one embodiment, individual lines of weakness 242 preferably include living hinges or other suitable features for increasing the flexibility of frame 220.

Dressing 200 preferably is a single unit that includes plural location options for retaining an anatomical sensor. According to one embodiment, the anatomical sensor may include electromagnetic spectrum sensor 1000 or another sensor for sensing over time a change of body structure, e.g., infiltration and extravasation. Preferably, individual fittings, e.g., first fitting 230a or second fitting 230b, provide alternate location options for coupling electromagnetic spectrum sensor 1000 to dressing 200. The location option that is most suitable is preferably selected based on one or more factors including: (i) location of the insertion site S; (ii) orientation of cannula 20; (iii) avoiding movement of cannula 20 or electromagnetic spectrum sensor 100 due to pulling or snagging tubing 32 or lead 1010; and (iv) comfort of the patient. Dressing 200 is advantageous at least because the most suitable of plural location options for coupling electromagnetic spectrum sensor 1000 is preferably selected.

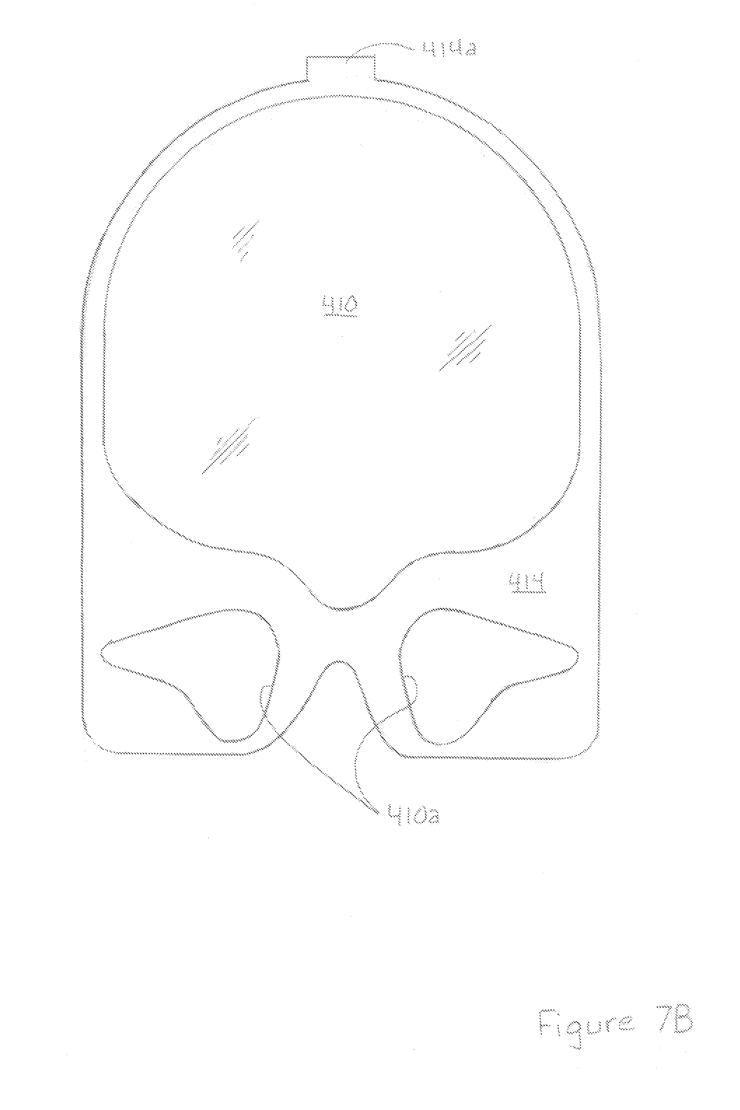

FIGS. 6A and 6B show embodiments of a dressing that include (i) a contamination barrier overlying the insertion site S; and (ii) different dressings 300a (FIG. 6A) and 300b (FIG. 6B) for locating electromagnetic spectrum sensor 1000 (not shown in FIG. 6A or 6B) to sense if fluid is infusing the perivascular tissue P around cannula 20. As compared to dressing 200, which includes a plurality of individual fittings at alternate location options on frame 220, dressings 300a and 300b separately provide different locations for a fitting 330 relative to a guide 314. Accordingly, one or the other of dressings 300a and 300b, rather than one or the other of first and second fitting 230a and 230b on dressing 200, may be selected for coupling electromagnetic spectrum sensor 1000 at the most suitable location option.

Dressings 300a and 300b preferably each include a pane 310, a frame 320 and fitting 330 that are functionally similar to, respectively, pane 210, frame 220 and first or second fitting 230a and 230b. Accordingly, dressings 300a and 300b preferably each provide a contamination barrier that is substantially impervious to solids, liquids, microorganisms and/or viruses, but which may be semi-permeable to allow air or vapor to pass, thus permitting the epidermis E to breathe. Pane 310 is preferably transparent or translucent to visible light for viewing the insertion site S. Frame 320 preferably maintains the shape of pane 310 while dressing 300a or dressing 300b is laid over the insertion site S. And a first arrangement of fitting 330 preferably retains electromagnetic spectrum sensor 1000 relative to dressing 300a or dressing 300b for monitoring an intravascular infusion by cannula 20, and a second arrangement of fitting 330 preferably releases electromagnetic spectrum sensor 1000 from the first arrangement.

Frame 320 preferably has greater resistance to deformation than does pane 310. Accordingly, frame 320 may maintain the shape of pane 310 while dressing 300a or dressing 300b is laid over the insertion site S. According to one embodiment, frame 320 entirely cinctures pane 310. According to other embodiments, frame 320 may (i) partially cincture pane 310; (ii) extend from a peripheral portion of pane 310 toward an interior portion of pane 310; (iii) extend from the interior portion toward the peripheral portion; (iv) be spaced from the peripheral portion; or (v) include a combination of (i)-(iv). Frame 320 preferably includes polyvinyl chloride, polyethylene, polypropylene, or another suitable material that is relatively rigid with respect to pane 310. For example, frame 320 may include a pad of polyvinyl chloride foam. Frame 320 may be opaque, but is preferably transparent or translucent to visible light for viewing the epidermis E in the vicinity of the insertion site S. Preferably, frame 320 absorbs or blocks the transmission of radiation having the same wavelength as emitted electromagnetic radiation 1002, e.g., near infrared radiation. Thus, according to one embodiment, the epidermis E that underlies frame 320 may be optically visible and shielded from ambient near-infrared radiation.

Dressing 300a and dressing 300b preferably are independent units that separately include different locations for retaining an anatomical sensor. Preferably, dressing 300a includes fitting 330 at a first location relative to guide 314, e.g., on the right-hand side of guide 314, and dressing 300b includes fitting 330 at a second location relative to guide 314, e.g., on the left-hand side of guide 314. Accordingly, the most suitable one of dressing 300a or dressing 300b preferably is selected based on one or more factors including: (i) location of the insertion site S; (ii) orientation of cannula 20; (iii) avoiding movement of cannula 20 or electromagnetic spectrum sensor 100 due to pulling or snagging tubing 32 or lead 1010; and (iv) comfort of the patient. Independent dressings 300a and 300b are advantageous at least because a choice is available for how an anatomical sensor is located relative to cannula 20.

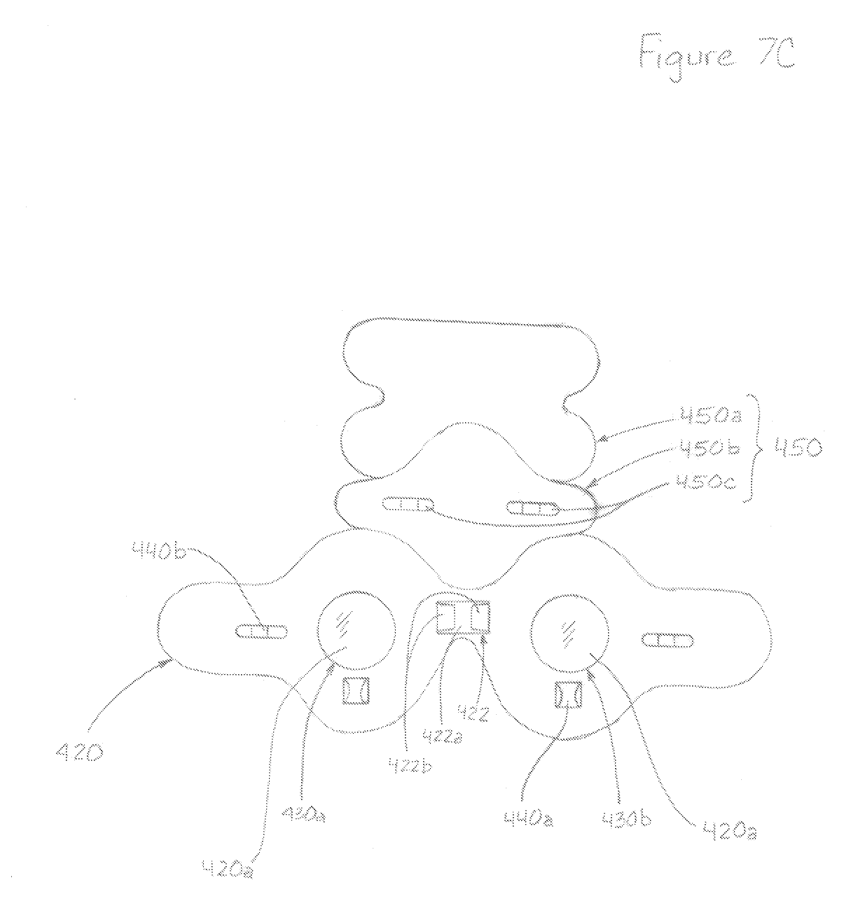

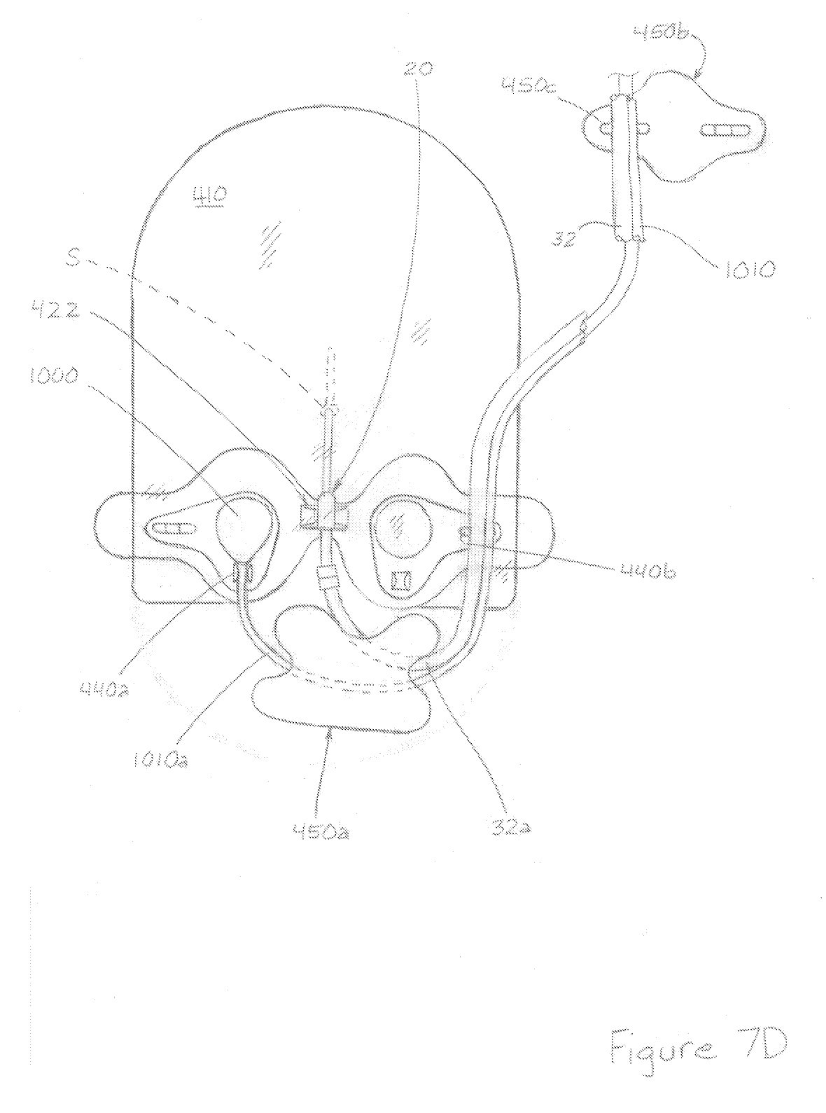

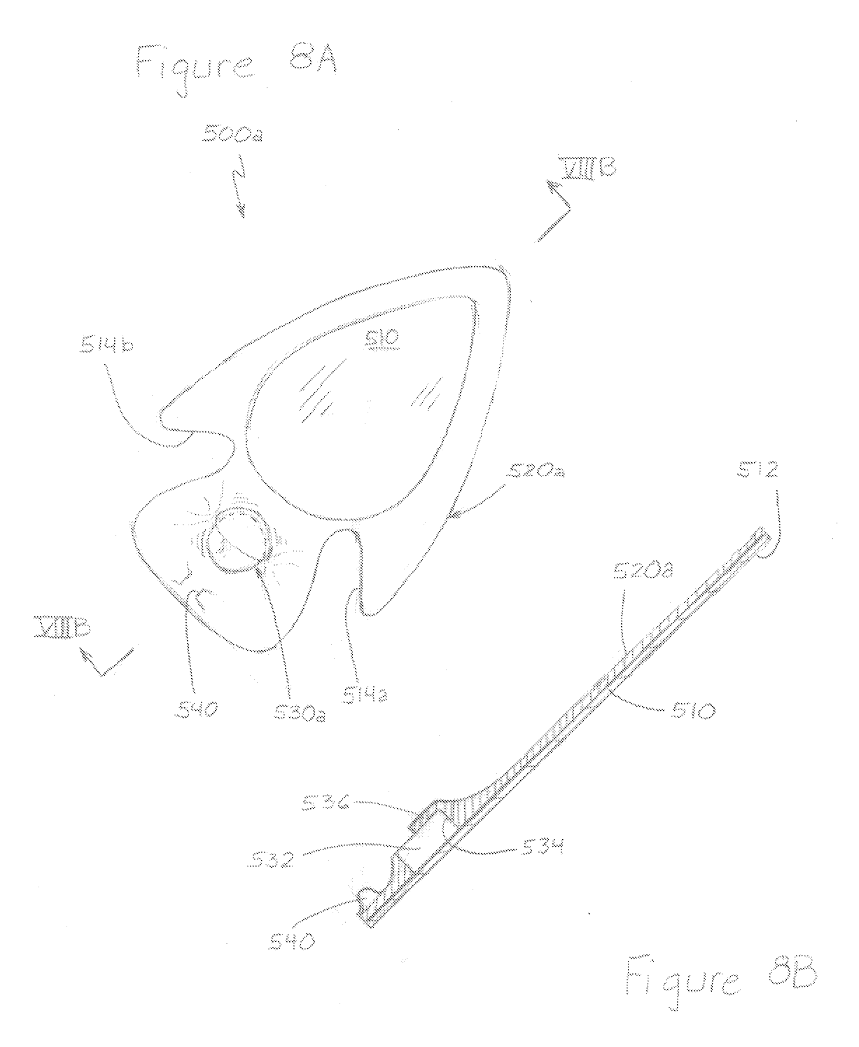

FIGS. 7A-7D show an embodiment of a dressing 400 that includes (i) a frame 420 that relatively positions electromagnetic spectrum sensor 1000 and cannula 20; and (ii) a contamination barrier that overlies the insertion site S and frame 420. The contamination barrier preferably is substantially impervious to solids, liquids, microorganisms and/or viruses, and may be semi-permeable to allow air or vapor to pass for permitting the epidermis E to breathe. The contamination barrier also preferably includes a pane 410 that is transparent or translucent to light in the visible portion of the electromagnetic spectrum for viewing the insertion site S. Pane 410 preferably includes a polyurethane film or another suitable material and/or construction for providing a contamination barrier that may be transparent or translucent.

An adhesive 412 preferably bonds the contamination barrier to the epidermis E (not indicated in FIGS. 7A-7D). Preferably, adhesive 412 includes an acrylic adhesive that is suitable for contact with the epidermis E or another medical grade adhesive that is biocompatible according ISO 10993 and/or USP Class VI. Adhesive 412 may be applied to the contamination barrier on the entire surface that confronts the epidermis E, or adhesive 412 may be omitted from one or more portions of the surface. For example, adhesive 412 may be omitted from a first area 412a on pane 410 in the vicinity of the insertion site S or from a second area 412b on pane 410 that may facilitate pulling pane 410 from the epidermis E. Preferably, the first or second areas 412a and 412b may be identified, e.g., with printing on pane 410. Also, the strength of the bond between pane 410 and the epidermis E may vary according to different embodiments of dressing 400. For example, stronger or more adhesive 412 may be used for coupling dressing 400 to relatively robust skin, e.g., adult skin, and weaker or less adhesive 412 may be used for coupling dressing 400 to relatively delicate skin, e.g., pediatric skin. Preferably, a removable backing sheet (not shown) preserves adhesive 412 until the contamination barrier is ready to be laid over the insertion site S and frame 420.

Referring particularly to FIG. 7B, a framework 414 preferably supports pane 410 while being laid over the insertion site S. Preferably, framework 414 includes paper or another suitable material that has greater resistance to deformation than does pane 410 but is flexible enough to conform to the contours of the epidermis E. Accordingly, framework 414 preferably maintains the approximate shape of the outer peripheral edge of pane 410 and of any apertures 410a (two are shown in FIGS. 7A, 7B and 7D) while the contamination barrier is being laid over the insertion site S and frame 420. According to one embodiment of dressing 400, a coupling between pane 410 and framework 414 is preferably relatively weaker than the bond between pane 410 and the epidermis E. Accordingly, framework 414 may be released after pane 410 bonds to the epidermis E. Preferably, a tab 414a facilitates pulling framework 414 from pane 410.

Frame 420 preferably has greater resistance to deformation than does pane 410. Preferably, frame 420 preferably includes polyvinyl chloride, polyethylene, polypropylene, or another suitable material that is relatively rigid with respect to pane 410. For example, frame 420 may include a pad of polyvinyl chloride foam. Frame 420 preferably distributes forces, e.g., due to pulling or snagging lead 1010, over an area of the epidermis E that is larger than that overlaid by sensor surface 1000a.

Frame 420 preferably links cannula 20 and electromagnetic spectrum sensor 1000. Preferably, frame 420 includes (i) a mount 422 for cooperatively engaging cannula 20; and (ii) at least one fitting—a first fitting 430a and a second fitting 430b are shown in FIGS. 7A, 7C and 7D—for coupling with electromagnetic spectrum sensor 1000. Accordingly, frame 420 preferably includes a link for establishing and maintaining a positional relationship between cannula 20 and electromagnetic spectrum sensor 1000. According to one embodiment, mount 422 preferably includes a base 422a and one or more resilient projections 422b extending from base 422a. Preferably, base 422a includes an interface for coupling mount 422 with frame 420, e.g., via an adhesive, and projection(s) 422b resiliently capture a portion of cannula 20. Therefore, mount 422 preferably establishes and maintains a positional relationship between cannula 20 and frame 420. Preferably, individual fittings, e.g., first fitting 430a or second fitting 430b, may be comparable to the fittings discussed above regarding dressing 200 and therefore each may retain electromagnetic spectrum sensor 1000. Therefore, each individual fitting preferably establishes and maintains a positional relationship between electromagnetic spectrum sensor 1000 and frame 420. Thus, according to one embodiment, frame 420, mount 422, and first fitting 430a or second fitting 430b preferably link cannula 20 and electromagnetic spectrum sensor 1000 by establishing and maintaining their relative positional relationship.

Referring particularly to FIG. 7C, frame 420 preferably prevents contiguous engagement between electromagnetic spectrum sensor 1000 and the epidermis E. Preferably, a barrier layer 420a extends across the pocket of individual fittings, e.g., first fitting 430a and second fitting 430b, and is interposed between sensor surface 1000a and the epidermis E in the first arrangements of the individual fittings. Barrier layer 420a may be the same material as pane 410 or another material that is substantially impervious to solids, liquids, microorganisms and/or viruses, and substantially transparent to emitted and received electromagnetic radiation 1002 and 1004.

Strain relief devices preferably redirect forces from electromagnetic spectrum sensor 1000 to dressing 400. Preferably, individual fittings, e.g., first fitting 430a or second fitting 430b, each include a set of strain relief devices that contiguously engage lead 1010 in the first arrangement. According to one embodiment, each set of strain relief devices preferably includes a first fixture 440a and a second fixture 440b. Individual fixtures 440a or 440b preferably each include a plurality of posts separated by a gap that is smaller than the diameter of lead 1010 and/or the diameter of tubing 32. Accordingly, lead 1010 and/or tubing 32 may be retained by a resilient interference fit between a pair of posts that preferably limit lateral and/or axial movement of lead 1010 or tubing 32 relative to frame 420.

Preferably, first and second fixtures 440a and 440b are disposed on opposite sides of mount 422. Each of FIGS. 7A, 7C and 7D indicate only one of two pairs of fixtures that are shown. In the first arrangement, first fixture 440a preferably retains lead 1010 proximate a first one of the first and second fittings 430a and 430b, and second fixture 440b preferably retains lead 1010 and tubing 32 proximate a second one of the first and second fittings 430a and 430b. First fixture 440a of first fitting 430a is shown on the left-hand side of mount 422 as viewed in FIG. 7D and second fixture 440b of first fitting 430a is shown on the right-hand side of mount 422 as viewed in FIG. 7D. According to one embodiment, first fixture 440a preferably cooperates with lead 1010 to eliminate or at least minimize rotation of electromagnetic spectrum sensor 1000 with respect to first fitting 430a, and second fixture 440b preferably establishes a first bight 1010a and a second bight 32a for lead 1010 and tubing 32, respectively.

A method of implementing dressing 400 will now be discussed with reference to FIG. 7D. Cannula 20 is inserted at insertion site S in a typical manner. Preferably, frame 420 is bonded to the epidermis E (not indicated) with projection(s) 422b of mount 422 engaging a portion of cannula 20. Pane 410 and framework 414 preferably are overlaid on frame 420 with apertures 410a cincturing first fitting 430a, second fitting 430b, and first and second fixtures 440a and 440b. Preferably, adhesive 412 bonds pane 410 to the epidermis E and framework 414 is separated from pane 410. Adhesive 412 preferably also adheres pane 410 over the portion of cannula 20 that is engaged by mount 422 so that cannula 20 is coupled to frame 420. Tubing 32 is coupled with cannula 20 in a typical manner and preferably also engages second fixture 440b to form bight 32a. Preferably, electromagnetic spectrum sensor 1000 is coupled to an individual fitting, e.g., the fitting on the left-hand side of mount 422 as viewed in FIG. 7D, with lead 1010 engaging first fixture 440a. Lead 1010 preferably also engages second fixture 440b to form bight 1010a. Electromagnetic spectrum sensor 1000 is thereby coupled to frame 420. Preferably, a lead management system 450 limits the forces that may be transmitted to dressing 400 as a result of pulling or snagging tubing 32 or lead 1010. Lead management system 450 preferably bonds to the epidermis E, e.g., with an adhesive, and includes a patch 450a and a board 450b. According to one embodiment, patch 450a preferably is shaped and sized to overlay bights 32a and 1010a, and board 450b preferably includes at least one fixture 450c that is similar to second fixture 440b in construction and function. Preferably, board 450b is spaced from bights 32a and 1010a along the lengths of tubing 32 and lead 1010. According to one embodiment, frame 420, patch 450a and board 450b preferably share a similar construction and may be manufactured concurrently as a unit, which may then be separated when implementing dressing 400.

Removing dressing 400 preferably occurs after releasing electromagnetic spectrum sensor 1000 from the first and second fittings 430a and 430b. Preferably, pane 410 is peeled off beginning with second area 412b while wings 420b (two are indicated on FIG. 7A) are held to separate pane 410 from frame 420. Cannula 20 preferably is disengaged from mount 422 and extracted from the insertion site S, and frame 420 is peeled off the epidermis E. A barrier film such as Cavilon™, manufactured by 3M (St. Paul, Minn., USA), or another topical agent may be used when implementing dressing 400 for protecting the epidermis E from adhesive trauma due to peeling off pane 410 and/or frame 420.

Dressing 400 is advantageous at least because there is a link between cannula 20 and electromagnetic spectrum sensor 1000 when sensing if fluid is infusing the perivascular tissue P around cannula 20. Preferably, frame 420, mount 422, and individual fittings, e.g., first fitting 430a or second fitting 430b, establish and maintain a relative positional relationship that links cannula 20 and electromagnetic spectrum sensor 1000. Dressing 400 is also advantageous because a contamination barrier is implemented in a typical manner, e.g., overlying the insertion site S, and concurrently cooperates with the link between cannula 20 and electromagnetic spectrum sensor 1000.

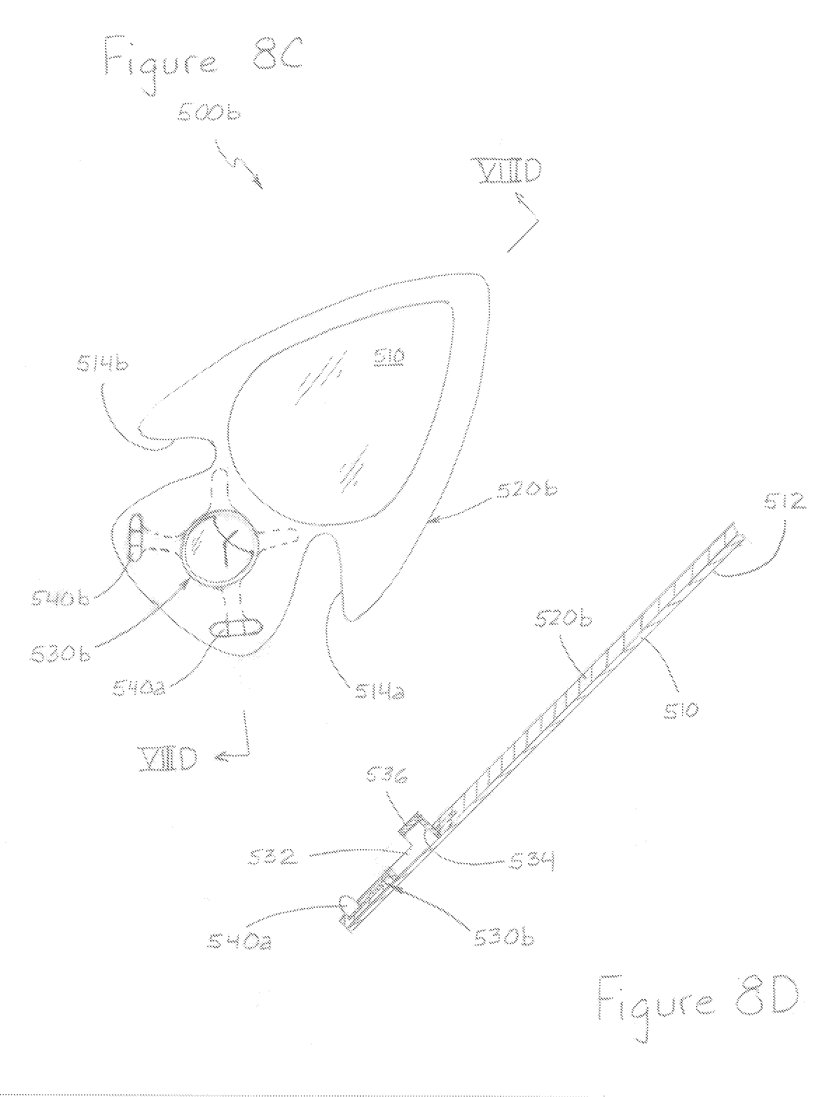

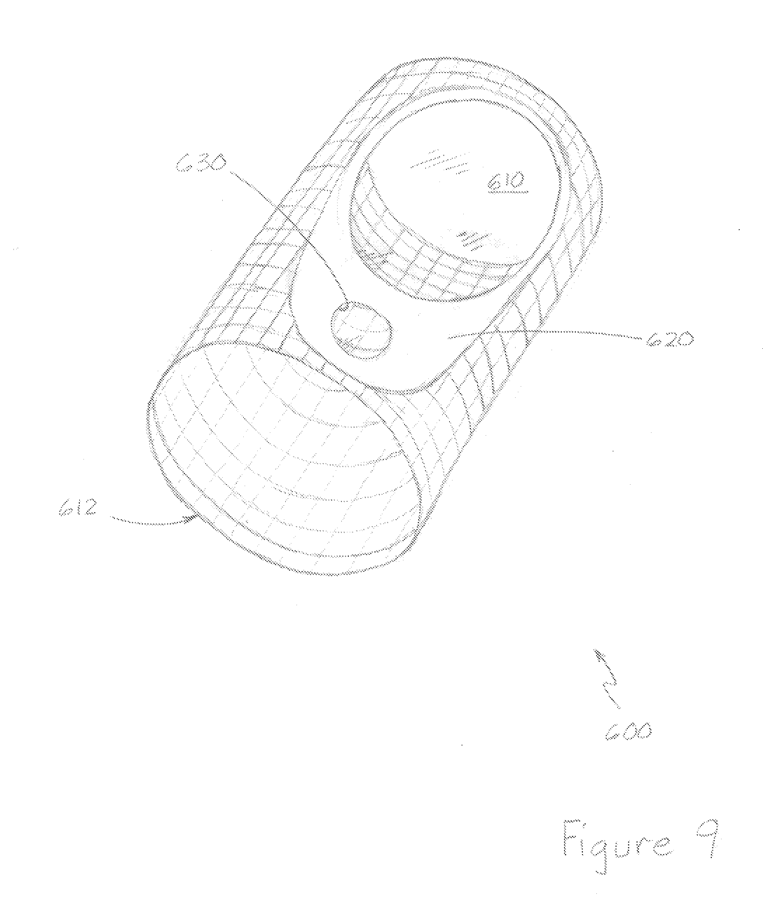

FIGS. 8A-8D show embodiments of dressings that include (i) a contamination barrier that overlies the insertion site S for cannula 20; (ii) a molded frame that locates electromagnetic spectrum sensor 1000 (not shown in FIGS. 8A-8D) to sense if fluid is infusing the perivascular tissue P around cannula 20; and (iii) a plurality of options for relatively locating electromagnetic spectrum sensor 1000 and cannula 20. Preferably, dressing 500a (FIGS. 8A and 8B) includes a first frame 520a that is integrally molded with a first fitting 530a, and dressing 500b (FIGS. 8C and 8D) includes a second frame 520b over-molding a second fitting 530b. The contamination barrier preferably is substantially impervious to solids, liquids, microorganisms and/or viruses, and may be semi-permeable to allow air or vapor to pass for permitting the epidermis E to breathe.

Employing molding to manufacture dressings 500a and 500b preferably reduces the number of independent components included in dressings 500a and 500b as compared to, for example, dressings 100, 200, 300a/300b and 400. Preferably, the phrase “independent component” as it is used herein refers to a single part that (a) has a substantially uniform composition; and (b) is coupled with other parts in an assemblage. Dressing 500a preferably reduces the number of independent components by at least two as compared to, for example, dressings 100, 200, 300a/300b or 400 because (i) first frame 520a and first fitting 530a may be formed as a single independent component, e.g., integrally molded with a homogeneous chemical compound, before assembling dressing 500a; and (ii) an adhesive for coupling first frame 520a with first fitting 530a may be eliminated. Dressing 500b preferably reduces the number of independent components by at least one as compared to, for example, dressings 100, 200, 300a/300b or 400 because an adhesive for coupling first frame 520a with first fitting 530a is eliminated. Preferably, further reductions are possible in the number of independent components included in dressings 500a and 500b as compared to dressings 200 or 400. For example, as compared to dressings 200 and 400, a further reduction of at least one additional independent component may be possible because first or second frames 520a or 520b and strain relief device(s) for lead 1010 may be formed as a single independent component, e.g., integrally molded with a homogeneous chemical compound, before assembling dressing 500a or 500b. And as compared to dressing 400, a yet further reduction of at least two additional independent components may be possible because (i) first or second frames 520a or 520b and a mount for cannula 20 may be formed as a single independent component, e.g., integrally molded with a homogeneous chemical compound, before assembling the dressing; and (ii) an adhesive for coupling the mount with first or second frames 520a or 520b may be eliminated. Thus, employing molding may reduce the number of independent components that preferably are included in dressings 500a and 500b.

Dressing 500a (or dressing 500b) preferably includes a pane 510, frame 520a (or frame 520b), and fitting 530a (or fitting 530b) that function similar to, for example, pane 310, frame 320 and fitting 330, respectively. Accordingly, pane 510 preferably is transparent or translucent to visible light for viewing the insertion site S; frame 520a (or frame 520b) preferably maintains the shape of pane 510 while dressing 500a (or dressing 500b) is laid over the insertion site S; and a first arrangement of fitting 530a (or fitting 530b) preferably retains electromagnetic spectrum sensor 1000 relative to dressing 500a (or dressing 500b) for monitoring an intravascular infusion by cannula 20 and a second arrangement of fitting 530a (or fitting 530b) preferably releases electromagnetic spectrum sensor 1000 from the first arrangement.

Pane 510 preferably uses an adhesive 512 to bond with the epidermis E in the vicinity of the insertion site S. Preferably, pane 510 includes a polyurethane film or another suitable material for providing a contamination barrier that may be transparent or translucent. Adhesive 512 preferably couples pane 510 to the epidermis E. Preferably, adhesive 512 includes an acrylic adhesive that is suitable for contact with the epidermis E or another medical grade adhesive that is biocompatible according ISO 10993 and/or USP Class VI. Adhesive 512 may be applied to pane 510 on the entire surface that confronts the epidermis E, or adhesive 512 may be omitted from one or more portions of the surface. Also, the strength of the bond between pane 510 and the epidermis E may vary according to different embodiments of the dressing. For example, stronger or more adhesive 512 may be used for coupling dressing 500a or dressing 500b to relatively robust skin and weaker or less adhesive 512 may be used for coupling dressing 500a or dressing 500b to relatively delicate skin.

Dressings 500a and 500b each preferably include a plurality of options for positioning or orienting the dressings on the epidermis E. Preferably, dressing 500a includes a first guide 514a at a first location relative to fitting 530a, e.g., on the right-hand side of fitting 530a as viewed in FIG. 8A, and a second guide 514b at a second location relative to fitting 530a, e.g., on the left-hand side of fitting 530a as viewed in FIG. 8A. Similarly, dressing 500b includes first guide 514a located on the right-hand side of fitting 530b as viewed in FIG. 8C, and second guide 514b located on the left-hand side of fitting 530b as viewed in FIG. 8C. The most suitable one of first guide 514a or second guide 514b preferably is selected based on one or more factors including: (i) location of the insertion site 5; (ii) orientation of cannula 20; (iii) avoiding movement of cannula 20 or electromagnetic spectrum sensor 1000 due to pulling or snagging tubing 32 or lead 1010; and (iv) comfort of the patient. According to one embodiment, individual guides 514a and 514b preferably include a notch or some other feature of dressing 500a or 500b that may be sized or shaped to receive a portion of cannula 20 (not shown in FIGS. 8A-8D). According to another embodiment, individual guides 514a and 514b preferably include a mount (not shown) for cooperatively engaging cannula 20. Alternate first and second guides 514a and 514b are advantageous at least because a choice is available for how electromagnetic spectrum sensor 1000 is located relative to cannula 20.

First and second frames 520a and 520b preferably have greater resistance to deformation than does pane 510. Accordingly, individual frames, e.g., first frame 520a or second frame 520b, may maintain the shape of pane 510 while dressing 500a or dressing 500b is laid over the insertion site S. First and second frames 520a and 520b preferably are formed as single independent components, e.g., integrally molded with a homogenous chemical compound, rather than being built-up as a laminate. Preferably, individual frames, e.g., first frame 520a or second frame 520b, include polydimethylsiloxanes or another suitable material for molding the frames. Advantageously, dressings 500a and 500b preferably resist absorbing fluids as compared to typical woven or fabric dressings.

First and second fittings 530a and 530b preferably are capable of retaining electromagnetic spectrum sensor 1000. Preferably, individual fittings, e.g., first fitting 530a or second fitting 530b, each include a pocket 532, a wall 534, and a tab 536. Pocket 532 preferably receives electromagnetic spectrum sensor 1000 (not shown in FIGS. 8A-8D) in the first arrangement. Preferably, pane 510 extends across pocket 532 and is interposed between sensor surface 1000a and the epidermis E in the first arrangement of the individual fittings. According to one embodiment, wall 534 preferably includes a plurality of individual segments disposed partially around pocket 532. Preferably, at least one tab 536 projects from wall 534 and overlies a portion of electromagnetic spectrum sensor 1000 in the first arrangement. Elastic deformation of wall 534 or tab 536 preferably permits electromagnetic spectrum sensor 1000 to snap-in to pocket 532 in the first arrangement and to snap-out from pocket 532 in the second arrangement. According to one embodiment, tab 536 preferably biases sensor surface 1000a toward the epidermis E by contiguously engaging electromagnetic spectrum sensor 1000 in the first arrangement. According to other embodiments, individual fittings may include a latch, a cap, a resilient element, or another suitable device which, in a first arrangement, retains electromagnetic spectrum sensor 1000 in pocket 532 and preferably biases sensor surface 1000a toward the epidermis E, and in a second arrangement, releases electromagnetic spectrum sensor 1000 from the first arrangement so as to permit movement out of pocket 532.

Dressings 500a and 500b preferably maintain an approximately consistent positional relationship between electromagnetic spectrum sensor 1000 and the perivascular tissue P. According to an embodiment of dressing 500a, frame 520a preferably distributes forces acting on electromagnetic spectrum sensor 1000 due to, e.g., pulling or snagging lead 1010, over an area of the epidermis E that is larger than that overlaid by sensor surface 1000a. Preferably, one or more arms 538 (four are shown in FIG. 8C) are coupled with wall 534 according to an embodiment of dressing 500b. Arm(s) 538 preferably extend away from pocket 532, e.g., beyond an area of the epidermis E that is overlaid by sensor surface 1000a in the first arrangement of fitting 530b. Accordingly, forces acting on electromagnetic spectrum sensor 1000 due to, e.g., pulling or snagging lead 1010, may be distributed by arm(s) 538 and frame 520b over an area of the epidermis E that is larger than that overlaid by sensor surface 1000a. Dressings 500a and 500b therefore preferably enhance an approximately consistent positional relationship between electromagnetic spectrum sensor 1000 and the perivascular tissue P when sensing infiltration or extravasation.

Strain relief devices preferably redirect forces from lead 1010 to dressing 500a or dressing 500b. Preferably, first frame 520a or second fitting 530b include at least one strain relief device that contiguously engages lead 1010 in the first arrangement. First frame 520a and a strain relief device 540 (FIGS. 8A and 8B) preferably are formed as a single independent component, e.g., integrally molded with a homogeneous chemical compound, before assembling dressing 500a. Second fitting 530b and first and second fixtures 540a and 540b (FIGS. 8C and 8D) preferably are formed as a single independent component, e.g., integrally molded with a homogeneous chemical compound, before assembling dressing 500b. According to an embodiment of dressing 500b, portions of first and second fixtures 540a and 540b preferably are exposed with respect to frame 520b. Preferably, strain relief device 540, first fixture 540a, and second fixture 540b each include a plurality of posts separated by a gap that is smaller than the diameter of lead 1010. Accordingly, lead 1010 may be retained by a resilient interference fit between a pair of posts that preferably limit lateral and/or axial movement of lead 1010 relative to frame 520a or frame 520b.

Molding during manufacturing of dressing 500a and 500b preferably includes at least one of (i) integrally molding a single independent component that fulfills more than one role in an assemblage; or (ii) over-molding a first independent component with another independent component in an assemblage. Preferably, first frame 520a is integrally molded with wall 534 and tab 536 as an independent component included in dressing 500a. Roles including maintaining the shape of pane 510 and retaining/releasing electromagnetic spectrum sensor 1000 are therefore fulfilled by a single independent component in dressing 500a. According to an embodiment of dressing 500a, strain relief device 540 preferably also is integrally molded with first frame 520a as an independent component included in dressing 500a. Accordingly, the additional role of limiting relative movement of lead 1010 is also fulfilled by a single independent component in dressing 500a. According to an embodiment of dressing 500b, preferably an initial shot in a multi-shot mold forms a first independent component and a subsequent shot in the multi-shot mold assembles dressing 500b, including the independent component formed with the initial shot. Preferably, second frame 520b over-molds second fitting 530b in dressing 500b. For example, wall 534 and tab 536 preferably are integrally molded with second fitting 530b as an independent component before being over-molded with second frame 520b. According to embodiments of dressing 500b, first fixture 540a and/or second fixture 540b preferably also are integrally molded with second fitting 530b as an independent component before being over-molded with second frame 520b. Employing molding in manufacturing dressings 500a and 500b is advantageous at least because fewer independent components are preferably assembled as compared to, for example, dressings 100, 200, 300a/300b or 400.

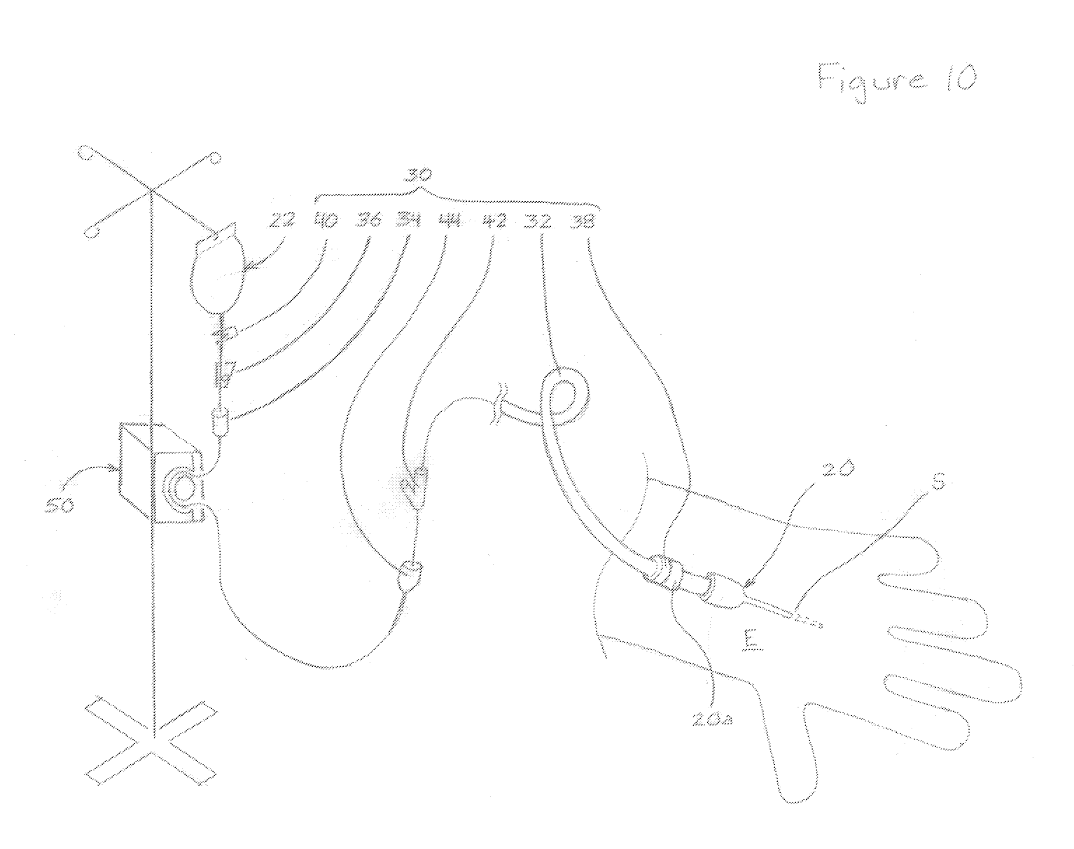

FIG. 9 shows an embodiment of a dressing 600 that includes (i) a contamination barrier that overlies the insertion site S for cannula 20; (ii) a frame that locates electromagnetic spectrum sensor 1000 (not shown in FIG. 9) to sense if fluid is infusing the perivascular tissue P around cannula 20; and (iii) a resilient band coupling the barrier and the frame with the epidermis E. The contamination barrier preferably is substantially impervious to solids, liquids, microorganisms and/or viruses, and may be semi-permeable to allow air or vapor to pass for permitting the epidermis E to breathe.

Preferably, dressing 600 includes a pane 610, a frame 620, and a fitting 630 that function similar to, for example, pane 310, frame 320 and fitting 330, respectively. Accordingly, pane 610 is preferably transparent or translucent to visible light for viewing the insertion site S. Frame 620 preferably maintains the shape of pane 610 while dressing 600 is laid over the insertion site S. A first arrangement of fitting 630 preferably retains electromagnetic spectrum sensor 1000 relative to dressing 600 for monitoring an intravascular infusion by cannula 20, and a second arrangement of fitting 630 preferably releases electromagnetic spectrum sensor 1000 from the first arrangement.

As compared to dressings 100, 200, 300a/300b, 400 and 500a/500b, which include an adhesive coupling with the epidermis E, dressing 600 preferably includes a non-adhesive coupling with the epidermis E. Preferably, a mesh band 612 is coupled to the peripheral edge of frame 620 so as to form a loop or band for cincturing a patient's limb. Mesh band 612 is preferably resilient for maintaining contiguous engagement of dressing 600 with the epidermis E. Accordingly, dressing 600 may be advantageous when it is preferable that the contamination barrier and/or fitting 630 not adhere to the epidermis E in the vicinity of the insertion site S.

While the present invention has been disclosed with reference to certain embodiments, numerous modifications, alterations, and changes to the described embodiments are possible without departing from the sphere and scope of the present invention, as defined in the appended claims. For example, embodiments of dressings that couple electromagnetic spectrum sensor 1000 and the epidermis E may be separate from a barrier film that may also be coupled to the epidermis E. Advantageously, such dressings may be applicable with a variety of independent contamination barriers and/or cannula mounts. Accordingly, it is intended that the present invention not be limited to the described embodiments, but that it has the full scope defined by the language of the following claims, and equivalents thereof.

What is claimed is:

1. A dressing for coupling a near-infrared sensor and an epidermis, the near-infrared sensor being configured to monitor an intravascular infusion, the dressing comprising:
a fitting including a wall at least partially defining a pocket, the fitting having (i) a first arrangement configured to retain the near-infrared sensor in the pocket and (ii) a second arrangement configured to release the near-infrared sensor from the first arrangement; and
a frame being coupled to the fitting and configured to overlay an area of the epidermis that is larger than that overlaid by the fitting.

2. The dressing of claim 1, comprising an adhesive configured to couple the frame and the epidermis.

3. The dressing of claim 1, comprising a film occluding the pocket.

4. The dressing of claim 3 wherein the film is approximately transparent to near-infrared signals configured to be emitted and detected by the near-infrared sensor.

5. The dressing of claim 3 wherein the film is configured to substantially prevent passage of solids, liquids, microorganisms, and viruses.

6. The dressing of claim 1 wherein the fitting is relatively more resistant to deformation than the frame.

7. The dressing of claim 1 wherein the fitting includes a flange projecting from the wall.

8. The dressing of claim 7, comprising an adhesive coupling the flange and the frame.

9. The dressing of claim 1 wherein the fitting consists of a first generally homogeneous material, the frame consists of a second generally homogeneous material, and the first generally homogeneous material is different from the second generally homogeneous material.

10. The dressing of claim 9, comprising a film occluding the pocket.

11. The dressing of claim 10 wherein the film consists of the second generally homogeneous material.

12. The dressing of claim 1 wherein the frame partially encapsulates the fitting.

13. The dressing of claim 1 wherein the fitting comprises an arm being coupled with the wall and extending away from the pocket.

14. The dressing of claim 13 wherein the frame at least partially encapsulates the arm.

15. The dressing of claim 1 wherein the fitting comprises a strain relief device configured to contiguously engage a lead of the near-infrared sensor in the first arrangement.

16. The dressing of claim 15 wherein the strain relief device is at least partially exposed with respect to the frame.

17. The dressing of claim 1 wherein the frame comprises a notch configured to receive a hub of a cannula administering the intravascular infusion.
